(19) United States
(12) Patent Application Publication
(10) Pub. No.: US 2021/0278866 A1
(43) Pub. Date: Sep. 9, 2021

(54) TEMPERATURE REGULATION OF PROSTHETIC LIMBS

(71) Applicants: Piper LALLA, Bow, NH (US); Ava R. CONLON, Bow, NH (US)

(72) Inventors: Piper LALLA, Bow, NH (US); Ava R. CONLON, Bow, NH (US)

(21) Appl. No.: 17/189,949

(22) Filed: Mar. 2, 2021

Related U.S. Application Data
(60) Provisional application No. 62/984,566, filed on Mar. 3, 2020.

(51) Int. Cl. G05D 23/20; A61F 2/58; A61F 2/64; A61F 2/66

(52) U.S. Cl. CPC G05D 23/2033; A61F 2/581; A61F 2/582; A61F 2002/6614; A61F 2/64; A61F 2/66; A61F 2/588

(57) ABSTRACT

The present application relates heating and cooling systems for prosthetic limbs, and to the use of such systems in prosthetic limbs. In one aspect, a system for temperature regulation of a prosthetic limb comprises a battery, an electrical connection, and at least one temperature regulation element.

### CROSS-REFERENCE TO RELATED APPLICATION

This application claims the benefit of and priority under 35 U.S.C. § 119(e) to U.S. Ser. No. 62/984,566, filed Mar. 3, 2020, the contents of which is hereby incorporated by reference in its entirety.

### FIELD OF THE INVENTION

This application relates to heating and cooling systems for prosthetic limbs.

### BACKGROUND

Prosthetic limbs are intended to restore function to a user by replacing a missing limb. Generally, prosthetic limbs are made of metal, which has high thermal conductivity and low heat capacity. Accordingly, prosthetic limbs often present challenges for temperature regulation which limit the activities of people who use prosthetic limbs. For example, in the winter, body heat can be lost through the prosthetic limb. Alternatively, in the summer, the prosthetic limb prevents heat from escaping causing discomfort to the user.

Heated sleeves have been proposed to address the challenge of temperature regulation of prosthetic limbs. A heated sleeve incorporates electrical heating elements into the sleeve and wraps around the outside of a prosthetic limb. However, heated sleeves are bulky and do not fit under layers of winter clothing because of the thickness of the sleeve and the size of the electrical components, including a battery pack.

### SUMMARY

In one aspect, a temperature regulation system for a prosthetic limb comprises a battery attached to the prosthetic limb, a temperature regulation element configured to attach to a thermally conductive component of a prosthetic limb and provide heating and cooling to the prosthetic limb, and an electrical connection connecting a battery, and the temperature regulation element.

In some embodiments, the temperature regulation element is selected from the group consisting of a resistive heater, a heat exchanger, a fluid heating or cooling system, a gel heating or cooling system, a chemical heating or cooling system, a fan, and combinations thereof.

In some embodiments, the temperature regulation element is selected from the group consisting of a heat exchanger, a fluid heating system, a fluid cooling system, and combinations thereof.

In some embodiments, the temperature regulation system further comprises a tube and a reservoir for a fluid.

In some embodiments, the temperature regulation system further comprises a pump.

In some embodiments, the temperature regulation system further comprises a network of temperature regulation elements configured to be attached at a plurality of points along a surface of the prosthetic.

In some embodiments, the temperature regulation system further comprises a control system configured to maintain the prosthetic limb at approximately a constant temperature.

In some embodiments, the control system comprises a user control.

In some embodiments, the control system comprises a temperature sensor.

In some embodiments, temperature sensor is selected from a group consisting of infrared sensors, thermocouples, resistance temperature detectors, thermistors, and combinations thereof.

In some embodiments, the control system includes a regulator.

In some embodiments, the prosthetic limb is metallic.

In some embodiments, the battery is located near a socket of the prosthetic limb.

In some embodiments, the battery is located external to the prosthetic limb

In one aspect, a method of heating or cooling a prosthetic limb comprises attaching the temperature regulation system of the preceding embodiments to the prosthetic limb.

In some embodiments, the temperature regulation system maintains a temperature of the prosthetic limb between about 60-100° F.

In some embodiments, the temperature regulation system maintains a temperature of a socket of the prosthetic limb between about 90-93° F.

In one aspect a prosthetic limb comprises the temperature regulation system of the preceding embodiments.

### BRIEF DESCRIPTION OF THE DRAWINGS

FIG. 1 shows an exemplary schematic of a temperature regulation system embodiment.

FIG. 2 shows an exemplary photograph of a temperature regulation system embodiment.

FIG. 3 shows an exemplary schematic of a temperature regulation system embodiment with a fluid heating and cooling system.

### DETAILED DESCRIPTION

In one aspect, a system for temperature regulation of prosthetic limbs comprises a battery, an electrical connection, and at least one temperature regulation element. The temperature regulation system can provide heating of a prosthetic limb. The temperature regulation system can provide cooling of a prosthetic limb. The temperature regulation system can provide both heating and cooling of a prosthetic limb.

In certain embodiments, the temperature regulation system is small and lightweight and can be worn under layers of clothing. In some embodiments, the system is no larger than a cell phone, for example 0.5 in×5 in×3 in. In some embodiments, the system is rigid. In some embodiments the system is flexible. In some embodiments, the system includes an external pack, which can be attached to the body, for example, using a waistband or cuff for the arm or leg.

In some embodiments, the temperature regulation system is built into a prosthetic limb. In other embodiments, the temperature regulation system is an accessory that can be attached to and detached from a prosthetic limb. In some embodiments, the temperature regulation system is a permanent component of the prosthetic limb. In some embodiments, the temperature regulation system can be removed, for example, in seasons when heating and cooling is not necessary.

In some embodiments, the prosthetic limb is an upper limb or a lower limb. In some embodiments, a prosthetic limb includes a socket which acts as an interface between the user's limb and the prosthetic limb. In some embodiments, the socket comprises a metal or composite material. In some embodiments, the distal end of the prosthetic includes an accessory or tool which provides function to the user or allows the user to interact with the world. In some embodiments, the system is used for an upper limb prosthesis. In some embodiments, an upper limb prosthesis comprises a socket, a metal hook or a hand. In some embodiments, an upper limb prosthesis comprises a mechanical hand, terminal device (hook, etc.), elbow, shoulder, or combinations thereof. In some embodiments, mechanical hands are powered or non-powered. In some embodiments, the system is used for a lower limb prosthesis. In some embodiments, a lower limb prosthesis comprises a socket, mechanical joint (i.e., knee), a foot, or a combination thereof. In some embodiments, mechanical joints are powered or non-powered.

In some embodiments, the prosthetic limb comprises a metal, polymer, composite material, or combinations thereof. In some embodiments, the prosthetic limb comprises a material that conducts heat sufficiently to distribute heat from the temperature regulation system over the prosthetic limb. Non-limiting examples of metals include aluminum, aluminum alloys, titanium, titanium alloys, stainless steel, and combinations thereof. Non-limiting examples of composites include carbon fiber and fiberglass. Non-limiting examples of polymers include silicone, urethane, and thermoplastic gels. In some embodiments, a silicone or thermoplastic elastomer gel is located at the interface between the user's skin and the prosthesis.

In some embodiments, a temperature regulation element can provide heating of the prosthetic limb. In other embodiments, the temperature regulation system comprises a temperature regulation element that provides heating of the prosthetic limb (i.e., a heating element). In some embodiments the temperature regulation element comprises a resistive heater, a heat exchanger, a fluid heating system, a gel heating system, or a chemical heating system.

In some embodiments, a temperature regulation element can provide cooling of the prosthetic limb. In other embodiments, the temperature regulation system comprises a temperature regulation element that provides cooling of the prosthetic limb (i.e., a cooling element). In some embodiments, the temperature regulation element comprises a fan that directs hot or cold air toward the prosthesis. In some embodiments, the fan cools skin directly through ports or windows in the prosthesis. In some embodiments, the fan cools a fluid (e.g., water or gel) that circulates through the prosthesis by a small pump as part of a fluid heating or cooling system. In some embodiments, the fan is on-board the prosthesis. In some embodiments, the fan is external to the prosthesis. In some embodiments, the temperature regulation element comprises a plurality of fans. In some embodiments the temperature regulation element comprises a fluid cooling system, a gel cooling system, or a chemical cooling system.

In some embodiments, a temperature regulation element can provide both heating and cooling of the prosthetic limb. In other embodiments, the temperature regulation system comprises a first temperature regulation element that provides cooling of the prosthetic limb (i.e., a cooling element) and a second temperature regulation element that provides heating of the prosthetic limb (i.e., a heating element).

In some embodiments, the temperature regulation elements are attached to thermally conductive materials on the prosthetic limb. In some embodiments, the heating elements are attached to thermally conductive materials that direct heating or cooling to the skin of the user. In some embodiments, the placement of the system is determined by a prosthetist, a practitioner of prosthetics, who is responsible for design and fitting of the prosthesis. In some embodiments, the thermally conductive materials include an accessory or tool at the distal end of the prosthetic. In some embodiments, the thermally conductive materials comprise metal.

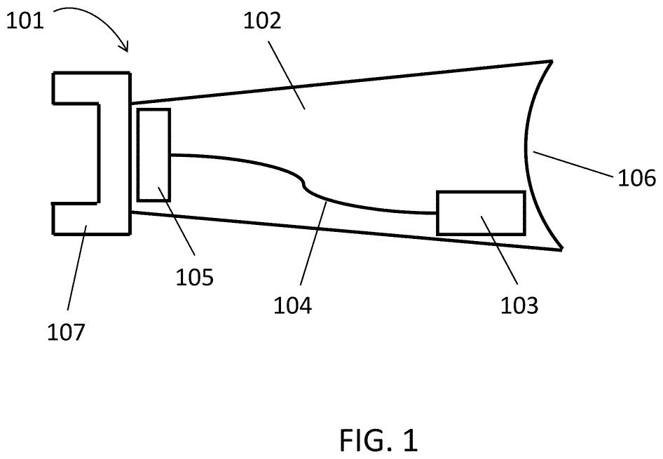

FIG. 1 shows an exemplary prosthetic limb 102 having a temperature regulation system. In some embodiments, the prosthetic limb 102 includes a socket 106 and a distal end 107 with a tool. In some embodiments, the temperature regulation system includes a battery pack 103 attached near the socket 106 of the prosthetic limb 102. In some embodiments, the battery pack is attached to the prosthetic limb by a clip. In some embodiments, the temperature regulation elements 105 are attached to the distal end 107 of the prosthetic limb 102 and heat the prosthetic limb. In some embodiments the temperature regulation element 105 is a heating pad. In some embodiments, the temperature regulation elements 105 are attached to thermally conductive components of the prosthesis, for example, metal components. In some embodiments, the temperature regulation elements 105 are attached to the battery pack 103 by an electrical connection 104. In some embodiments, the electrical connection is a wire.

In some embodiments, the temperature regulation system 101 is powered by a battery. In some embodiments, the battery is a rechargeable battery. In some embodiments, the temperature regulation system is powered by a solar panel element. In some embodiments, the battery or solar power element is incorporated into the prosthetic limb. In some embodiments, the battery or solar power element is removable or modular. In some embodiments the battery is located on the prosthetic limb. In some embodiments, the battery is attached to the prosthesis by a clip, laminated into the prosthetic limb, or thermoformed directly into the prosthetic limb. In some embodiments, the battery is located in an internal compartment of the prosthetic limb. In some embodiments, the internal compartment includes removable door or slide closure. In some embodiments, a battery housing is external.

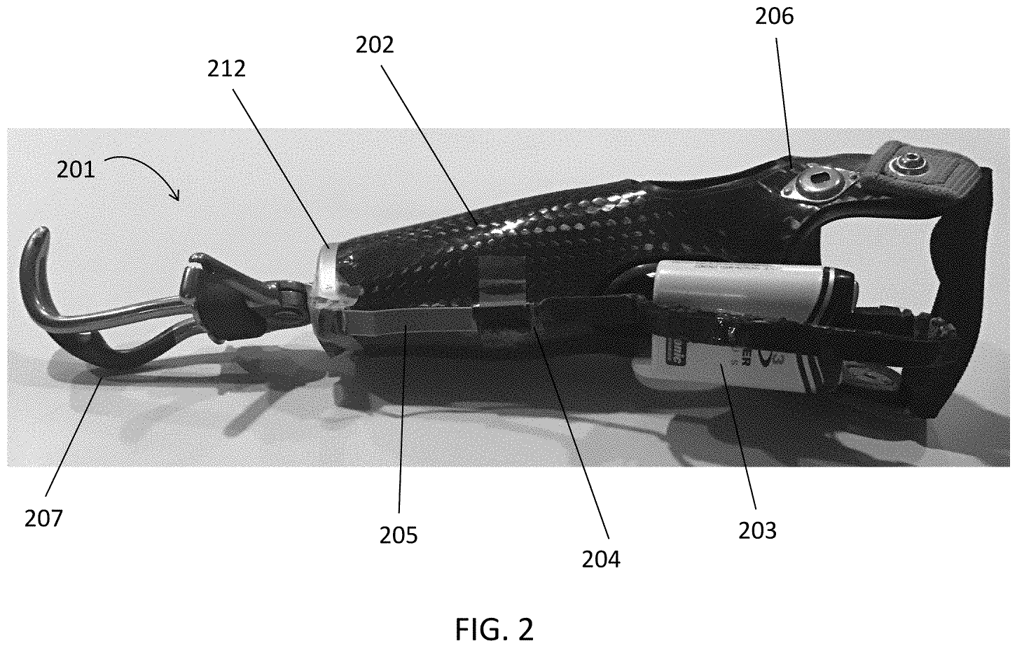

FIG. 2 shows a photograph of an exemplary temperature regulation system 201. In this photograph, the prosthetic limb 202 includes a carbon fiber socket 206 and a distal end 207 with a metal hook. In this photograph, a battery pack 203 is attached to the prosthetic limb 202 near the socket 206 of the prosthetic limb. In this photograph, a heating pad 205 is attached to a metal component 212 near the distal end 207 of the prosthetic limb. The heating pad 205 is connected to the battery 203 by a wire 204. In this example, the heating pad was modified from a boot heater (Hottronic Footwear). The heating pad includes a resistive heating element with a water-resistant coating. In this example, the battery is a four cell lithium ion battery.

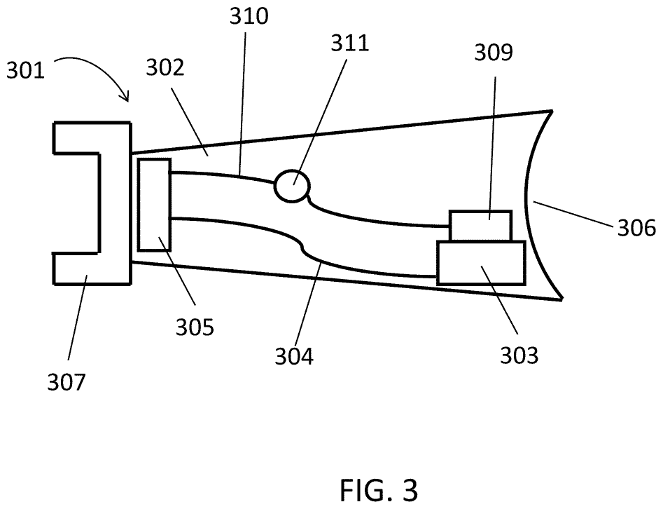

FIG. 3 shows a schematic of an exemplary temperature regulation system 301 with a fluid heating and cooling system. In some embodiments, the fluid is stored in a reservoir 309. In some embodiments, the fluid is stored within the plumbing or tubes of the system. In some embodiments, the temperature regulation system 301 includes a tube 310 to deliver a fluid (e.g., a liquid or a gel) to the temperature regulation element 305 and a reservoir 309 to store the fluid. In some embodiments, the temperature regulation system includes a pump 311 that moves the fluid throughout the temperature regulation system (e.g., between the reservoir 309 and the temperature regulation element 305) to heat or cool the prosthetic limb 302. In some embodiments, the battery pack 303 and the reservoir 309 are attached to the prosthetic limb 302 near the socket 306 of the prosthetic limb. In some embodiments, the temperature regulation element 305 is attached near the distal end 307 of the prosthetic limb and connected to the battery 303 by a wire 304. In some embodiments, the temperature regulation element 305 is also connected to the reservoir 309 by a tube 310. In some embodiments, the tube 310 includes a pump 311 to move the fluid from the reservoir 309 to the heating pad 305 or from the heating pad 305 to the reservoir 309. In some embodiments, the reservoir 309 is located on or in the prosthetic limb. In other embodiments, the reservoir 309 is external to the prosthetic. In some embodiments, the reservoir 309 can be worn on the torso or limb of the user. In some embodiments, the pump 311 is located in or on the prosthetic limb. In other embodiments, the pump 311 is external to the prosthetic. In some embodiments, the pump 311 can be worn on the torso or limb of the user

In some embodiments, the temperature regulation system keeps the surface of the prosthetic socket at a standard skin temperature. In some embodiments, the temperature regulation system keeps the surface of the prosthetic socket between about 87-90° F., 90-93° F., 93-96° F., or 96-99° F. In some embodiments, the temperature regulation system keeps the surface of the prosthetic socket between about 87-99° F. In some embodiments, the temperature regulation system keeps the surface of the prosthetic socket between about 87-96° F. In some embodiments, the temperature regulation system keeps the surface of the prosthetic socket between about 90-99° F. In some embodiments, the temperature regulation system keeps the surface of the prosthetic socket between about 90-96° F. In some embodiments, the temperature regulation system keeps the surface of the prosthetic socket between about 90-93° F. In some embodiments, the temperature regulation system keeps the prosthetic limb at a temperature between about 60-100° F. In some embodiments, the temperature regulation system keeps the prosthetic limb at a temperature between about 60-70° F., 70-80° F., 80-90° F., or 90-100° F. In some embodiments, the temperature regulation system keeps the prosthetic limb at a temperature between about 70-90° F. In some embodiments, the temperature regulation system keeps the prosthetic limb at a temperature between about 60-80° F. In some embodiments, the temperature regulation system keeps the prosthetic limb at a temperature between about 80-100° F. In some embodiments, the temperature of the prosthetic socket and the prosthetic limb is determined by the prosthetist.

In some embodiments, the temperature regulation system comprises a control system configured to maintain the prosthetic at a constant temperature. In some embodiments, the control system comprises a temperature sensor, a regulator, and a user control. Non-limiting examples of temperature sensors include infrared sensors, thermocouples, resistance temperature detectors, and thermistors. In some embodiments, the user control is located on the prosthetic limb. In some embodiments, the user control is external to the prosthesis to allow the user to more easily adjust the settings of the temperature regulation system, for example the temperature. In some embodiments, the user control is external to the prosthetic limb and communicates with the prosthetic limb with Bluetooth or Wi-Fi. In some embodiments, the user control is the user's cellular phone or other personal electronic device. In some embodiments, the control system maintains the prosthetic limb at a temperature of about 60-100° F., 60-70° F., 70-80° F., 80-90° F., or 90-100° F. and prevents the thermal regulation element from becoming too hot or too cold.

In some embodiments, the system for temperature regulation comprises an accessory. In some embodiments, the accessory can attach to the distal end of the prosthetic limb. Non-limiting examples of accessories include hooks, clips, or claws configured to hold a ski pole.

In some embodiments, the system for temperature regulation includes an external pack. In some embodiments, the external pack contains a battery, a control system, a user control, a reservoir of a fluid heating and cooling system, a pump of a fluid heating and cooling system, and/or combinations thereof. In some embodiments, the external pack is worn on the torso (e.g., using a waistband) or around a limb (e.g., using a cuff for an arm or leg).

In some embodiments, a method of heating or cooling a prosthetic limb comprises attaching a temperature regulation system to a prosthetic limb. In some embodiments, the temperature regulation system maintains the temperature of the prosthetic limb between about 60-100° F., 60-70° F., 70-80° F., 80-90° F., or 90-100° F.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

1. A temperature regulation system for a prosthetic limb comprising,
a battery attached to the prosthetic limb,
a temperature regulation element configured to attach to a thermally conductive component of a prosthetic limb and provide heating and cooling to the prosthetic limb, and
an electrical connection connecting a battery and the temperature regulation element.

2. The temperature regulation system of claim 1, wherein the temperature regulation element is selected from the group consisting of a resistive heater, a heat exchanger, a fluid heating or cooling system, a gel heating or cooling system, a chemical heating or cooling system, a fan, and combinations thereof.

3. The temperature regulation system of claim 2, wherein the temperature regulation element is selected from the group consisting of a heat exchanger, a fluid heating system, a fluid cooling system, and combinations thereof.

4. The temperature regulation system of claim 1, further comprising a tube and a reservoir for a fluid.

5. The temperature regulation system of claim 1, further comprising a pump.

6. The temperature regulation system of claim 1, further comprising a network of temperature regulation elements configured to be attached at a plurality of points along a surface of the prosthetic.

7. The temperature regulation system of claim 1, further comprising a control system configured to maintain the prosthetic limb at approximately a constant temperature.

8. The temperature regulation system of claim 7, wherein the control system comprises a user control.

9. The temperature regulation system of claim 7, wherein the control system comprises a temperature sensor.

10. The temperature regulation system of claim 9, wherein the temperature sensor is selected from a group consisting of infrared sensors, thermocouples, resistance temperature detectors, thermistors, and combinations thereof.

11. The temperature regulation system of claim 7, wherein the control system includes a regulator.

12. The temperature regulation system of claim 1, wherein the prosthetic limb is metallic.

13. The temperature regulation system of claim 1, wherein the battery is located near a socket of the prosthetic limb.

14. The temperature regulation system of claim 1, wherein the battery is located external to the prosthetic limb

15. A method of heating or cooling a prosthetic limb comprising
attaching the temperature regulation system of claim 1 to the prosthetic limb.

16. The method of claim 15, wherein the temperature regulation system maintains a temperature of the prosthetic limb between about 60-100° F.

17. The method of claim 15, wherein the temperature regulation system maintains a temperature of a socket of the prosthetic limb between about 90-93° F.

18. A prosthetic limb comprising the temperature regulation system of claim 1.
